(19) United States
(12) Patent Application Publication
(10) Pub. No.: US 2025/0114043 A1
(43) Pub. Date: Apr. 10, 2025

(54) METHODS AND DEVICES FOR MAPPING PHYSIOLOGICAL SENSOR DATA

(71) Applicant: Impact Vitals,Inc., Vienna, VA (US)

(72) Inventors: John Jedziniak, Greenwood Village, CO (US); Abhishek Jaiantilal, Highlands Ranch, CO (US)

(73) Assignee: Impact Vitals, Inc, Vienna, VA (US)

(21) Appl. No.: 18/729,152

(22) PCT Filed: Jul. 13, 2023
(86) PCT No.: PCT/US23/27686

§ 371 Date: Jul. 15, 2024

Related U.S. Application Data
(60) Provisional application No. 63/526,648, filed on Jul. 13, 2023.

(51) Int. Cl. A61B 5/00; A61B 5/0295; A61B 5/346

(52) U.S. Cl. CPC A61B 5/7264; A61B 5/0295; A61B 5/346; A61B 5/725

(57) ABSTRACT

Physiological data from a wide variety of physiological sensors and sensor types may be mapped to values that are independent of the type of sensor or equivalent to a value that would be produced by another sensor.

### RELATED APPLICATION

The present application is a US national stage application of PCT Application number PCT/US23/27686 (the “686 Application”). The present application claims priority to the '686 Application and U.S. Provisional Application No. 63/526,648 filed Jul. 13, 2023 (the “'648 Application). The present application incorporates by reference the entire disclosures of the '686 and '648 Applications as if each were set forth in full herein.

### BACKGROUND

This section introduces aspects that may be helpful to facilitate a better understanding of the described disclosures. Accordingly, the statements in this section are to be read in this light and are not to be understood as admissions about what is, or what is not, in the prior art.

When different physiological data of an individual is collected using a variety of different sensor types made by a variety of different manufacturers there is a need to ensure that the different data that is collected by the different types of sensors can be interpreted correctly. For example, different sensor manufacturers may use light emitting diodes (LEDs) that emit different wavelengths of light and may also use different signal processing to sense and capture physiological waveforms (e.g., obtain a signal representing a PPG waveform). The differences in wavelengths and processing may cause subtle, yet important, differences in the shape of the physiological waveforms (i.e., in the physiological data) that may be obtained by a particular sensor.

Still further, some sensors may incorporate different reception technology. For example, one type of reception technology comprises a photodiode that detects light from an LED that reflects off the tissue of an individual while another technology comprises a photodiode that detects light from an LED as the light passes through the tissue of the individual. Again, these differences may also contribute to important differences in the physiological data obtained by a particular sensor.

Furthermore, physiological differences between signals (e.g., photoplethysmogram (PPG) waveforms) obtained for the same individual at different body locations (e.g., different tissues) using the same type of sensors may also result in detectable differences in the shape of a physiological signal, and in the corresponding physiological data.

Collectively, all of the above different types of physiological data sensed by sensors may be referred to herein simply as “different physiological data” or “different data”.

To date, interpretation of such different physiological data requires disparate computational processing and data, including, as an example, collection of a statistically significant amount of data from human trials, and the related re-training and re-validation of machine learning (ML) models, for each different sensor, which can be very costly, and resource and time intensive.

Accordingly, it is desirable to provide improved methods and devices that correctly interpret different physiological data from a wide variety of sensor types.

### BRIEF DESCRIPTION OF DRAWINGS

FIG. 1 depicts a simplified block diagram of an innovative method for mapping historical, different physiological data from a plurality of different physiological sensor types to correctly interpret physiological values of an individual according to embodiments of the disclosure.

FIG. 2 depicts a simplified block diagram of an innovative method for mapping current, different physiological data from a plurality of different physiological sensor types to correctly interpret physiological values of an individual according to embodiments of the disclosure.

FIG. 3 depicts exemplary intermediate values representing PPG waveforms that may be generated by embodiments of the present disclosure.

FIGS. 4A and 4B depict PPG waveforms according to embodiments of the present disclosure.

FIG. 5 depicts a series of waveforms according to embodiments of the present disclosure.

FIGS. 6A to 6D depict exemplary PPG waveforms according to embodiments of the present disclosure.

FIGS. 7A to 7C depict exemplary PPG validation waveforms according to embodiments of the present disclosure.

### SUMMARY

The inventors describe a number of innovative methods for mapping physiological data. For example, one exemplary method may comprise: electronically receiving different physiological data, the data representing one or more of sensed photoplethysmography (PPG) or electrocardiogram (ECG) waveforms; electronically portioning the different physiological data into segments; electronically generating one or more sensor-dependent values representing each segment; electronically assigning each of the sensor-dependent values to an intermediate value; and further electronically assigning each of the intermediate values to a value that represents one or more physiological states or levels or physiological signals.

In such an exemplary method the different physiological data may comprise: (a) physiological signal data sensed by sensors that are classified differently; and/or (b) physiological signal data sensed by sensors positioned at different specific locations on an individual's body; and/or (c) physiological signal data sensed by sensors that use different specific, operating characteristics; and/or (d) physiological signal data sensed by sensors that output data using different, specific formats or protocols.

Further, such an exemplary method may further comprise one or more of the following steps: (1) electronically portioning the different physiological data into segments further comprises electronically portioning the different physiological data into segments of equal length; (2) electronically portioning the different physiological data into segments further comprises electronically portioning one or more of the portioned segments into a physiological signal segment having a different length than another portioned segment; (3) electronically filtering one or more of the PPG or ECG waveforms that cannot be represented by a two or higher dimensional value when the intermediate values comprise a two or higher dimensional value; (4) electronically determining a relative indication of one or more physiological states of an individual based on a two or higher dimensional intermediate values, when, again, the intermediate values comprise a two or higher dimensional value; (6) applying a rotational or slope adjustment to the PPG or ECG waveforms to account for physiological changes; and (7) electronically filtering one or more of the PPG or ECG waveforms that cannot be represented by an N-dimensional value when the generated intermediate values do not comprise N-dimensional values.

In addition to innovative methods, the present disclosure also provides innovative electronic devices. One such electronic device for mapping physiological data may comprise: one or more electronic processors operable to execute instructions stored in one or more electronic memories to: electronically receive different physiological data, the data representing one or more of sensed photoplethysmography (PPG) or electrocardiogram (ECG) waveforms; electronically portion the different physiological data into segments; electronically generate one or more sensor-dependent values representing each segment; electronically assign each of the sensor-dependent values to an intermediate value; and further electronically assign each of the intermediate values to a value that represents one or more physiological states or levels or physiological signals.

In embodiments of such an exemplary electronic device the different physiological data may comprise: (a) physiological signal data sensed by sensors that are classified differently; (b) physiological signal data sensed by sensors positioned at different specific locations on an individual's body; (c) physiological signal data sensed by sensors that use different specific, operating characteristics; and/or (4 physiological signal data sensed by sensors that output data using different, specific formats or protocols.

Further, in such an exemplary electronic device the one or more electronic processors may be further operable to execute instructions stored in one or more electronic memories to (1) electronically portion the different physiological data into segments of equal length; (2) electronically portion the different physiological data into one or more physiological signal segments having a different length than another portioned segment; (3) electronically filter one or more of the PPG or ECG waveforms that cannot be represented by a two or higher dimensional value when the intermediate values comprise a two or higher dimensional value; (4) electronically determine a relative indication of one or more physiological states of an individual based on the two or higher dimensional value, when the intermediate values comprise a two or higher dimensional value; (5) apply a rotational or slope adjustment to the PPG or ECG waveforms to account for physiological changes; and/or (6) electronically filter one or more of the PPG or ECG waveforms that cannot be represented by an N-dimensional value when the generated intermediate values do not comprise N-dimensional values.

### DETAILED DESCRIPTION, WITH EXAMPLES

Exemplary embodiments of methods and devices for mapping different physiological data from a plurality of different physiological sensor types as well as from the same or similar sensors placed on different locations of an individual's body are shown by way of example in the drawings. Throughout the following description and drawings, like reference numbers/characters may refer to like elements.

It should be understood that although specific embodiments are discussed herein, the scope of the disclosure is not limited to such embodiments. On the contrary, it should be understood that the embodiments discussed herein are for illustrative purposes, and that modified and alternative embodiments that otherwise fall within the scope of the disclosure herein are contemplated.

As used herein, the words “comprising”, and any form thereof such as “comprise” and “comprises”; “having”, and any form thereof such as “have” and “has”; “including”, and any form thereof such as “includes” and “include”; are inclusive or open-ended and do not exclude additional, unrecited elements or process steps.

As used herein, the term “a” or “an” may mean “one”, but may also mean “one or more”, “at least one”, and “one or more than one” depending on the usage and context.

It should also be understood that one or more exemplary embodiments may be described as a process or method. Although a process/method may be described as sequential, it should be understood that such a process/method may be performed in parallel, concurrently or simultaneously. In addition, the order of each step within a process/method may be re-arranged. A process/method may be terminated when completed and may also include additional steps not included in a description of the process/method.

As used herein, the term “and/or” includes any and all combinations or permutations of one or more of the associated listed items.

It should be understood that when one part or step in an innovative device or method is described or depicted as being “connected” to another part or steps, other parts or steps used to facilitate such a connection may not be described or depicted because such parts or steps are well known to those skilled in the art.

Yet further, when one part or step of a device or method is described or depicted as being connected to another part or step in a figure it should be understood that practically speaking such a connection may comprise (and many times will comprise) more than one physical connection or processing step.

It should be noted that the devices, as well as any components, or elements thereof, illustrated in the figures are not necessarily drawn to scale, and need not be representative of an actual shape or size and need not be representative of any actual device. Rather, the devices, components and elements are drawn so as to help explain the features, functions and processes of various exemplary embodiments of the described disclosure.

Relatedly, to the extent that any of the figures or text included herein depicts or describes operating parameters it should be understood that such information is merely exemplary and non-limiting and is provided to enable one skilled in the art to practice an exemplary embodiment of the disclosure.

Where used herein, the letter “n” or “N” may denote the last step or component of one or more steps or components (e.g., electronic memories 1a to 1n, or sensors A to N).

As used herein the term “physiological state” means a condition that can be inferred from physiological data. Examples of physiological states include dehydration, heat stress, and temperature shock. For purposes of prevention, diagnosis, and treatments, it is useful to sense and collect (i.e., monitor) data that indicates the existence of potentially harmful physiological states, and to sense and collect data regarding the severity of such states.

As used herein, the terms “embodiment” or “exemplary” refer to a nonlimiting example of the present disclosure.

As used herein, the term “physiological signal segment” or “signal segment” refers to a sequence of physiological values (e.g., PPG waveform values) derived from physiological signal data that may encompass any number of values.

As used herein, the term “type of sensor” or “types of sensors” includes each of the following: (1) a sensor having a specific classification (e.g., transmissive or reflective PPG sensors), (2) a sensor positioned at a specific location on an individual's body (e.g., on a wrist or forehead), (3) a sensor having a specific operating characteristic (e.g., infrared or another wavelength or frequency) and (4) a sensor that functions to output a physiological signal in a specific format or protocol. Accordingly, the phrase “different type(s) of sensor(s)” or “different sensor types” means for purposes of this disclosure a sensor or sensors whose specific classification, or location on a different part of an individual's body, or opening characteristic or data format or protocol is different than another sensor (or sensors).

As used herein the phrase “different physiological data” includes physiological signal data that may have been sensed by sensors (1) that are classified differently (e.g., transmissive or reflective PPG sensors), and/or (2) positioned at different specific locations on an individual's body (e.g., on a wrist or forehead), and/or (3) that derive data using different specific, operating characteristics (e.g., wavelengths, frequencies), and/or (4) that output data using different, specific formats or protocols.

As used herein, the term “map”, “mapping” or “signal mapping” refers to the assignment of one signal segment derived from a first type of sensor during a given time period to another signal segment derived from a second type of sensor during the same time period.

As used herein the phrase electronic processors “operable to” (e.g., one or more electronic processors 2a to 2n, or 6a to 6n) or similar phrases means that the one or more electronic processors function to execute electronic instructions retrieved from their electronic memories (not shown in Figures) to complete one or more enumerated functions described herein of an innovative device(s) or one or more processes of an innovative method(s).

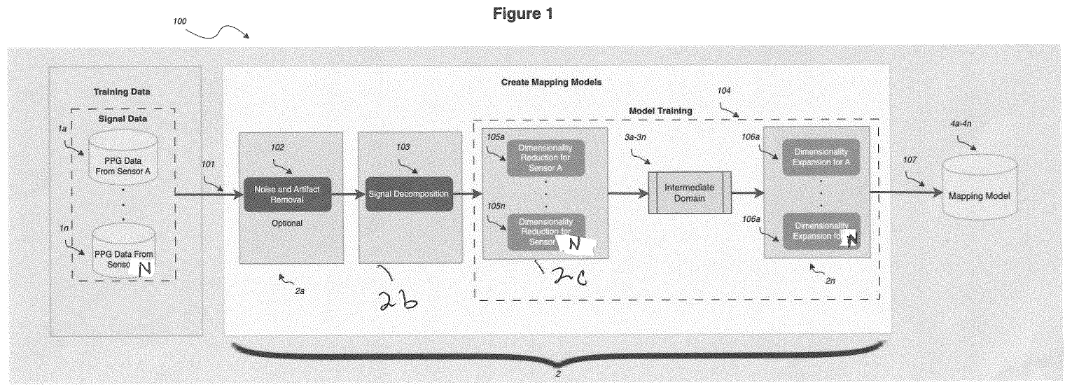

Referring now to FIG. 1 there is depicted a simplified block diagram of an innovative referential method 100 for mapping a wide variety of different physiological signal data according to an embodiment of the disclosure.

In step 101, a plurality of historical, different physiological data from one or more electronic storage devices 1a to 1n (databases, electronic servers) may be sent to, and received by, one or more electronic processors 2a to 2n which may be part of one or more electronic devices 2 such as an electronic server, PC and the like. As noted above such different physiological data may have been previously sensed and collected from a plurality of different types of sensors and then stored in storage devices 1a to 1n or may have currently been sensed and collected by different sensor types and then stored in storage devices 1a to 1n before being sent to processors 2a to 2n. In embodiments, the different physiological data may comprise a wide variety of sensed data, such as sensed heart pulse rates, blood pressures, PPG data, electrocardiogram (ECG or EKG) data, and the like. As further noted above, such different physiological data may comprise data sensed and collected from different parts of an individual's body by the same type of sensor (e.g., data representing PPG waveforms sensed from the same type of sensor located at different parts of an individual's body).

In more detail, the different physiological data may have been collected from two or more sensors in such a manner that the different physiological data was captured at the same time, accounting for differences in affecting mechanisms such as, but not limited to, sensor frequency and sensor location. For example, data representing physiological signals may be collected from an infrared (IR) PPG sensor placed on the left index finger of an individual while at the same time data representing physiological signals was collected from a green PPG sensor (i.e., a sensor that outputs a visible wavelength of light, e.g., in this case green light) placed on the right wrist of the same individual, with the two sets of data synchronized in time such that the PPG value time components reflect the blood flow changes caused by the same heartbeat during the same time period.

Continuing, upon receiving different physiological data from electronic storage devices 1a to 1n in step 101, the one or more electronic processors 2a to 2n involved in completing steps of method 100 may be operable to map a physiological signal segment derived from one sensor during a given time period to physiological signal segment derived from a second sensor during the same time period during a mapping process 104 represented by steps 105a to 105n, 106a to 106n and 107.

It is believed that the innovative mapping described herein provides a method of effectively and accurately interpreting physiological data from different types of sensors as well as reducing the complexity of computations required to ensure correct interpretations.

In embodiments, as an option, prior to the mapping process 104 (sometimes referred to as “training process”) the one or more electronic processors 2a to 2n may be operable to transform the different physiological data into an improved representation of such data by electronically adjusting the different physiological data by removing unwanted or undesirable electronic noise or artifacts (representing erroneous signals or data, collectively referred to as “corrupted data”) from the received different physiological data in step 102 using one or more analog or digital electronic filters or filtering processes, thereby increasing the accuracy of the received data, and eventually, any mapping process that may be based on the received data.

In more detail, corrupted data may be caused by different motions or positions (e.g., walking versus running versus repetitive motions, and/or standing versus sitting versus supine) made by the individuals connected to sensors that were involved in experiments or tests from which the different physiological data may have been derived. Accordingly, in step 102, the electronic processors 2a to 2n may be operable to adjust the different physiological data by electronically removing such corrupted data by electronically adding or subtracting stored compensation values (not shown), for example, in accordance with their stored instructions. Alternatively, instead of adjusting the data using processors 2a to 2n, the different data may be initially adjusted before being stored in electronic memories 1a to 1n by using accelerometers, multiple PPG sensors or other sensors (not shown) that are configured to remove the corrupted data introduced as the individuals were involved in different motions and positions to generate the referential different data.

Continuing, upon optionally electronically adjusting the different data in step 102 the different data may then be further transformed in step 103 by decomposing the data into one or more different physiological signal segments.

In an embodiment the one or more processors 2a to 2n may be operable to electronically portion the different data into physiological signal segments of equal length. As an alternative, the one or more processors 2a to 2n may be operable to electronically portion one or more of the portioned segments into a physiological signal segment having a different length than another portioned segment. The so portioned segments may be stored in an electronic storage device or memory (not shown).

In more detail, the different data comprises physiological data from different types off sensors (i.e., the data is “sensor-dependent”). In accordance with an embodiment of the invention, data from each of these different types of sensors is electronically portioned into segments and stored. For example, physiological data from a first sensor (sensor “A”) may be portioned into segments and stored and physiological data from a second, different type of sensor (sensor “B”) may also be portioned and stored. Likewise, data from each sensor “N” (where “N” indicates the last sensor) may be portioned and stored during step 103.

In an embodiment, each portioned segment from sensor A may be associated with a given or specific period of time. Similarly, each portioned segment from every other sensor (e.g., sensors B to N) may be associated with the same periods of time on a segment-by-segment basis. Thus, each portioned segment from one sensor (e.g., sensor A) may be “time-aligned” with a portioned segment from one or more additional sensors (e.g., B to N). By so time-aligning each portioned segment, a time-aligned portioned segment from one sensor (e.g., sensor A) may be mapped in steps 105a to 105n, 106a to 106n and 107 to a time-aligned portioned segment associated with the one or more additional sensors (e.g., sensors B to N).

For the reader's benefit, each of the sensors A to N comprise sensors that may be applied to the same individual during the same time period.

Using a PPG waveform as an example, in an embodiment, each portioned segment of a PPG waveform from sensor A (e.g., the IR sensor discussed above) may be associated with a given or specific period of time. Similarly, each portioned segment of a PPG waveform from one or more additional sensors (e.g., the green light sensor discussed above) may be associated with the same periods of time on a segment-by-segment basis. Thus, each portioned segment of a PPG waveform from a first sensor may be “time-aligned” with a portioned segment of a PPG waveform from a second sensor. By so time-aligning each portioned segment a time-aligned portioned segment from one sensor may be mapped in steps 105a to 105n, 106a to 106n and 107 to a time-aligned portioned segment associated with the one or more different sensors (i.e., the second sensor).

Of course, it should be understood that the sensors described above have been placed on the same individual and have been monitored to operate such that they begin their collection of physiological data from the same individual at the same time.

For ease of understanding values representing the time-aligned segments for a given sensor may be referred to hereafter as “first values” or “a set of first values” for a given sensor.

In an embodiment, the time aligned segments from step 103 may be inputted into the mapping process 104. In an embodiments, to complete the mapping process 104 the one or more processors 2a to 2n may be operable to receive varied input data representing heart waveforms (e.g., PPG, ECG waveforms) from steps 101 to 103, and then generate one or more values and mapping processes (sometimes referred to as “mapping training models) that may then be stored in electronic storage devices 4a to 4n during step 107, that can be used for later mapping of other signal data such as from real-time signal sources.

In an embodiment, the mapping process 104 involves the generation of intermediate and final values, and the comparison of those final values with known values. The process 104 may also include the adjustment of a set of values in order to generate final values that “best fit” or best represent a waveform.

The mapping process 104 may assign the value from a sensor to an intermediate value (which may be referred to as “forward mapping”) or from an intermediate value to a sensor (which may be referred to as “reverse mapping”). In an embodiment, the mapping process may comprise a generative artificial intelligence (AI) process that implements neural network processing with latent dimensions. Alternatively, the mapping process 104 may include a constrained optimization process that includes dimensionality reduction steps (e.g., Principal Component Analysis (PCA) or Fourier Analysis based processes).

During steps 105a to 105n of the exemplary mapping process 104 the one or more electronic processors 2a to 2n may be operable to assign each of the sensor-dependent, time-aligned, portioned segments of each sensor (i.e., a first sensor) and its associated first values to one or more sensor-independent, intermediate values. For ease of understanding the assigned, sensor-independent intermediate values may be referred to as “second values”, or “a second set of values”.

In embodiments, each second set of values assigned to a portioned segment may be represented by one or more values that are less complex than the first set of values associated with a portioned segment (i.e., the dimensions of the first set of values representing a portioned segment are estimated and then reduced to a second set of values). For example, a first set of values representing each sensor-dependent portioned segment from each sensor may be assigned to one or more sensor-independent, two-dimensional, intermediate value, to name just one example of an intermediate value. Collectively, a second set of values (i.e., one or more sensor-independent intermediate values) may be viewed as representing a time-aligned segment.

Accordingly, the mapping process 104 includes functions and steps that convert values representing each sensor-dependent segment into one or more sensor-independent intermediate values, for example (sometimes referred herein as “mapping process values”) which may be stored in electronic storage devices 4a to 4n.

In embodiments, because collectively-speaking, all of the time-aligned portioned segments may represent a PPG waveform derived from a given sensor, then, collectively, all of the so-assigned intermediate values also represent the same PPG waveform.

Furthermore, the mapping process may comprise neural network based processes that may generate interpolative, or even extrapolative, waveform values with a substantial degree of accuracy.

It should be noted the exemplary mapping process used in steps 105a to 105n to generate the sensor-independent intermediate values may comprise a constrained optimization process that includes a Fourier transformation process, for example, which reduces the dimensions (and thus computational complexity) of the first set of values.

Continuing, following the generation of the sensor-independent intermediate values (second set of values), in one embodiment such values may then be further transformed into a third set of values during steps 106a to 106n (i.e., a value that represents one or more physiological states or levels or physiological signals).

In an embodiment, during steps 106a to 106n the one or more processors 2a to 2n may execute stored instructions, retrieved from their memory (not shown) that comprise one or more mapping processes and mapping values to generate and assign a third set of values (e.g., PPG waveform values) that correspond to values that are estimated from the second set of values. In one embodiment, the mapping process may comprise a “best fit” process, where, for example, each set of third values are estimated or “fitted” to be close to the first values (using only the second values).

In more detail, an exemplary best fit process may comprise the step of determining the estimated difference between the third values and the first set of values using only the second set of values.

Said another way, the differences between the third set of values (output of step 107) and the first set of values (e.g., representing a PPG waveform) based on some metric, such as a mean squared error or absolute error. The closer the difference is to 0 the better the “fit”.

(sometimes referred to as determining a “loss function”) to minimize the difference between the two and may include the determination of other differences (e.g., a quadratic loss function (e.g., mean square error), binary cross-entropy loss, or hinge loss, among many others).

The best fit process implemented during steps 106a to 106n to generate the third set of values may further comprise a process that includes an inverse Fourier transformation process, for example, which expands the dimensions of the second set of values such that the dimension of the third set of expanded values matches the dimensions of the first set of values prior to steps 105a to 105n. Additionally, the best fit process ensures that the third set of values closely match the first set of values using only the second set of values as inputs. The first, second and third set of values may be stored in electronic storage devices 4a to 4n as part of one or more “models”.

Additionally, steps 105a to 105n and 106a to 106n ensures that all the time-aligned portioned segments that may represent a PPG sensor obtained from two or more input sensors collectively have similar, if not the same, intermediate values.

Said another way, during process 104, the time-aligned portioned segments that may represent a PPG waveform derived from a given sensor are associated with a first set of values, which are converted into intermediate values (a second set of values) comprising a reduced dimension during steps 105a to 105n. the second set of values may then be expanded into a third set of values that closely represent the same PPG waveform (the first set of values) during steps 106a to 106n. When time-aligned portioned segments that may represent a PPG waveform derived from two or more sensors are “available” (i.e., both sensors A and sensor B are their respective time aligned data are available, such that the data can be mapped to the same intermediate values as compared to when only one data from one sensor is available, then mapping may not be attempted) during steps 105a to 105n then the intermediate values (second set of values) may be very similar if not the same values with each other (i.e., for two time aligned segments from sensor A and one from sensor B, the intermediate values are ensured to be the same, or very close to each other in value). The mapping process values (i.e., first, second and third set of values) may be stored in electronic storage devices 4a to 4n as part of a “model”.

In even more detail, suppose α and [symbol] are two time-aligned portioned segments that represent a PPG waveform from two given sensors A and B respectively. We can use the nomenclature m( ) to represent a mathematical function “m” and m(x) to represent values obtained from the function m( ) with inputs x; additionally, if m( ) and n( ) are two functions and x is some input, then to evaluate m(n(x)), we first use x as an input to n( ) to get values n(x) and then use values n(x) as an input to m( ) to get values m(n(x)).

Further, suppose the process of reducing the dimensions of a value for sensor A during step 105a can be represented by the function f( ) while the process of expanding the dimensions of a value for sensor A during step 106a can be represented by the function f′( ), where f′( ) is the inverse function of f( ); an inverse function is the reverse of the given function and thus f′(f(x))=x for some input x. Function f( ) converts the first values α, from sensor A, into a second values f(α) that are sensor independent. Similarly, f( ) can take the second values f(α) and convert them into set of third values so that f′(f(α))=α.

Similarly, the process of reducing the dimensionality of a value for sensor B, during step 105b can be represented by the function g( ) and the process of expanding the dimensionality of sensor B during step 106b can be represented by the function g′( ), where g′( ) is the inverse function of g( ). Accordingly, function g′( ) can take the second values g( [symbol] ) and convert them into set of third values so that g′(g( [symbol] )= [symbol] . Additionally, it can be ensured that f(α)=g( [symbol] ), that is the second values are the same for both PPG waveforms α and [symbol] .

In an embodiment, the innovative best fit processes identify relationships between the values associated with sensors A and B (i.e., identify relationships between functions f( ), f( ), g( ), g′( ), such that g′(g( [symbol] ))= [symbol] , f(f(α))=α, and f(α)=g( [symbol] )). Additionally, when only segments from a single sensor are available, e.g., only α from sensor A is available, then the best fit processes identify relationships between the values derived from sensor A (i.e., identify relationships between functions f( ), f( ) such that f′(f(α))=α.).

In embodiments, the best fit processes may generate a set of parameters which may be used in conjunction with the first, second or third set of values and may be stored in electronic storage devices 4a to 4n as part of a model.

For example, this best fit process may involve a loss function, or error function, seeking to minimize the difference, and may include a quadratic loss function (e.g., mean square error), binary cross-entropy loss, or hinge loss, among many others. Note that for the sake of simplicity, we assumed that we can find the “best fit” parameters for functions f( ) and f( ) and similarly for g( ) and g′( ) such that f′(f(α))=α and g′(g( [symbol] ))= [symbol] , but in real world data we have to assume that such a perfect fit may not be possible and we may get an approximate fit where f′(f(α))=α+ε₁ and g′(g( [symbol] ))= [symbol] +ϵ₂, where ε₁ and ε₂ are very small numbers.

Expounding on the previous description, suppose α and [symbol] are two time-aligned portioned segments that represent a PPG waveform from two given sensors A and B respectively. Suppose we want to take the waveform a from sensor A and we want to transform it so that it matches [symbol] , we need to perform the operation g′(f(α)), where f(α) converts the first values α into intermediate (second values) that are sensor independent. Then we dimensionality expand using a g′( ) function that takes the second values f(α) and converts them into set of third values for sensor B. These third values are very close to if not equal to [symbol] . This process works because we ensured that f(α)=g( [symbol] ), that is the second values are the same for the first values of both α and [symbol] which are two time-aligned portioned segments that represent a PPG waveform from two given sensors A and B at the same time. Thus, substituting f(α)=g( [symbol] ) in g′(f(α)), we get g′(f(α))=g′(g( [symbol] ))= [symbol] . The function, g′(f( )) is performed using step 105a followed by step 106b with α, the time-aligned portioned segment representing a PPG waveform from sensor A, as first values to obtain [symbol] as output of step 104.

Still further examples may benefit the reader. Suppose one set of first values representing a PPG waveform from sensor A is available. In an embodiment, during steps 105a to 105n, the processors 2a to 2n may transform these first set of values to a second set of values utilizing a set of mapping process values that were previously stored (using the mapping model). Then, during steps 106a to 106n, the processors 2a to 2n may again transform these second set of values to a third set of values in a way that these third set of values closely represent the PPG waveform from sensor A (the first set of values), also utilizing the previously retained set of mapping processes. The best fit process in this case is to use the first set of values to generate a second set of values with reduced dimensions, and then a third set of values from the second set of values, in such a way that the third set of values closely match the first set of values. The retained set of mapping algorithm values are then adjusted based on the best fit process to minimize the difference in further calculations.

Suppose further that one set of first values representing a PPG waveform from sensor A and another set of first values representing a PPG waveform from sensor B are available and are time-aligned. In an embodiment, during steps 105a to 105n, the processors 2a to 2n may transform these first set of values to a second set of values. Additionally, steps 105a to 105n complete a best fit process that attempts to fit the second set of values representing the PPG waveform sensed by sensor A and the PPG waveform sensed by sensor B to be the same; this best fit process for matching the second set of values from multiple sensors is only used when time-aligned portioned segments of PPG waveforms from more than one sensor are available. Said another way, the best fit process also ensures that the second set of values for both PPG waveform from sensor A and PPG waveform from sensor B are the same (or very close to each other). This ensures that the mapping of both PPG waveforms from different sensors, the first set of values, to the same set of second values. Also, similar to the previous example, the mapping process values are adjusted during this process. In another embodiment, during steps 106a to 106n, the processors 2a to 2n may again transform these second set of values to a third set of values in a way that these third set of values closely represent the PPG waveform from sensor A or sensor B as desired (the first set of values) and are compared to known values for best fit processing and adjusting of mapping process values. Note that the same mapping process can be performed for more than two sensors and depends only on time-aligned portioned segments of PPG waveforms.

The mapping process 104 discussed herein is believed to help solve the challenges involved in interpreting different data from different sensor types. For example, if process 104 determines that the second set of values representing a PPG waveform sensed by sensor A closely aligns with second set of values representing a PPG waveform sensed by sensor B, then the third set of estimated values representing the PPG waveform represented by sensor B may be used to interpret (and reconstruct) the PPG waveforms from sensors A and B. Said another way, the PPG waveforms from sensor A may be mapped to the PPG waveforms of sensor B. In embodiments, by a similar process, the PPG waveforms from sensor B may be mapped to the PPG waveforms of sensor A and be used to interpret (and re-construct) the PPG waveforms from sensors B and A. Still further, in embodiments, heart waveforms (PPG, ECG waveforms) from one type of sensor may be mapped to the heart waveforms of another type of sensor by completing mapping process 104 to aid in the re-construction and interpretation of such heart waveforms.

Further, while the example above describes the mapping of waveforms between two sensors, this is merely exemplary. Alternatively, mapping may occur between one set of waveforms and another set of waveforms, for example.

In an embodiment, as noted previously the first, second and third sets of values may be stored in one or more electronic storage devices 4a to 4n during step 107. For a given set of sensors that originally generated the different data stored in devices 1a to 1n, the stored first, second and third set of values may comprise a set of values that may be used as one or more electronic processing “models” to correctly interpret and reconstruct physiological data (e.g., PPG, ECG waveforms) from a plurality (i.e., A to N) of different sensors. Further, the models stored in one or more electronic storage devices 4a to 4n may be used to estimate and predict one or more physiological states, such as dehydration or temperature shock to name just two of the many physiological states, as explained further herein and in co-pending U.S. application No. ______ the contents of which are incorporated in full herein.

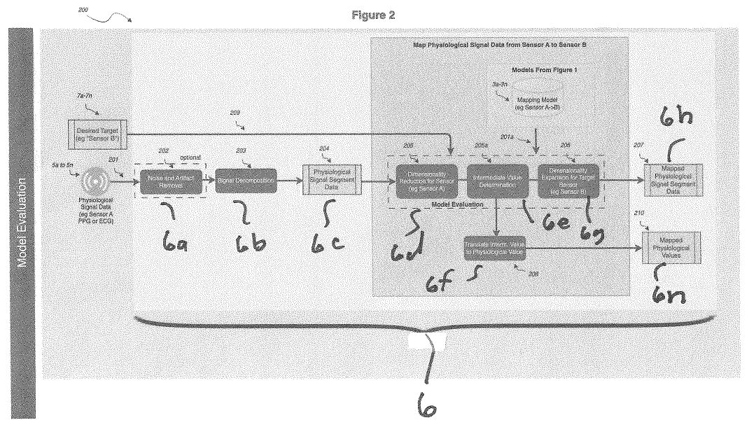

Referring now to FIG. 2 there is depicted a simplified block diagram of an innovative real-time process or method 200 for, among other things, estimating and predicting current values of different physiological levels and states. The inventors believe that such estimates may be critical in life-saving efforts as well as in personal, physiological monitoring and physical performance improvement.

In an embodiment, copies of some or all of the electronic instructions stored in the electronic memories of processors 2a to 2n, the intermediate values stored in storage devices 3a to 3n and the electronic processing model(s) stored in electronic storage devices 4a to 4n during step 201a may be electronically transferred to one or more electronic processors 6a to 6n that may be a component(s) of one or more user devices 6 (e.g., a device used by an individual interested in personal, physiological monitoring or a device that may be used by a medical professional, technician or hospital attendant such as a watch, mobile phone, laptop computer, PC or server(s)). Alternatively, similar instructions, values and models may be stored on processors 6a to 6n using other existing electronic means and processes.

Similar to the referential method 100 described herein, upon receiving current or stored, different physiological data from sensors 5a to 5n (e.g., PPG waveform data, ECG waveform data and other physiological data described herein) in step 201, and signal data that identifies the waveform of sensor to be mapped from target sensors 7a to 7n in step 209,

In an embodiment, method 200 may transform the physiological data from sensors 5a to 5n to one or more portioned segments representing a PPG waveform from sensor A.

In more detail, the one or more electronic processors 6a to 6n executing method 200 may be operable to retrieve instructions stored in their electronic memory (not shown) in order to transform the received, current different physiological data (collectively referred to as “current, different data”) into current mapped values, and/or into current physiological values including, but not limited to, hydration levels and/or heat levels using additional steps 207 (physiological signals are stored), 208 and 210 (physiological values are stored) described elsewhere herein as well in co-pending U.S. application No. ______ the contents of which are incorporated in full herein.

The one or more electronic processors 6a to 6n may be operable to electronically adjust the received current, different data into an improved representation of such data by optionally and electronically removing corrupted data from the received current different data in step 202, as described previously in step 102, to increase the accuracy of the received data, and eventually, current, estimated and/or predicted physiological values, for example.

Continuing, in step 203 the one or more electronic processors 6a to 6n may be further operable to decompose the current, different data received from sensors 5a to 5n into current, physiological signal segments as similarly described with respect to step 103 above.

In more detail, the one or more processors 6a to 6n may execute instructions stored in their electronic memories (not shown) to electronically portion the current, different data (that is sensor-dependent) into physiological signal segments of equal length. As an alternative, one or more of the portioned segments may have a different length. The so portioned segments may be stored in an electronic storage device or memory (not shown).

In an embodiment, each portioned segment from each sensor 5a to 5n may be associated with a given or specific period of time (e.g., a first portioned segment of sensor 5a, a first portioned segment of sensor 5b . . . and a first portioned segment of sensor 5n is associated with the same first time period; a second portioned segment of sensor 5a, a second portioned segment of sensor 5b . . . and a second portioned segment of sensor 5n is associated with the same second time period; . . . a last portioned segment of sensor 5a, a last portioned segment of sensor 5b . . . and a last portioned segment of sensor 5n is associated with the same last period).

Thus, each portioned segment from one sensor 5a to 5n may be “time-aligned” with a portioned segment from one or more different sensors 5a to 5n. By so time-aligning each portioned segment a time-aligned portioned segment from one sensor 5a to 5n may be mapped in steps 205, 205a and 206 to a time-aligned portioned segment associated with the one or more additional sensors 5a to 5n.

For the reader's benefit, each of the sensors 5a to 5n comprise sensors that may be applied to the same individual during the same time period.

Using a PPG waveform as an example, in an embodiment, each portioned segment of a PPG waveform from sensor 5a (e.g., the IR sensor discussed previously) may be associated with a given or specific period of time. Similarly, each portioned segment of a PPG waveform from each other, different sensor, such as sensor 5b (e.g., the green light sensor discussed above) may be associated with the same periods of time on a segment-by-segment basis. Thus, each portioned segment of a PPG waveform from a first sensor 5a may be “time-aligned” with a portioned segment of a PPG waveform from a second sensor, for example, sensor 5b. By so time-aligning each portioned segment a time-aligned portioned segment from one sensor 5a to 5n may be mapped in steps 205, 205a and 206 to a time-aligned portioned segment associated with the one or more different sensors 5a to 5n.

Again, it should be understood that the sensors 5a to 5n may be placed on the same individual and have been monitored to operate such that they begin their collection of physiological data from the same individual at the same time. In an embodiment, at least one sensor 5a to 5n is placed on an individual.

In an embodiment, one or more time aligned segments from previous steps may be inputted into the mapping process comprising steps 205, 205a, 206 (which may be referred to as a “current mapping process to distinguish it from mapping process 104 in method 100). For ease of understanding values representing the time-aligned segments for a given sensor stored during step 204 may be referred to hereafter as “first current values” or “a set of first current values” for a given sensor.

During step 205 of the exemplary mapping process the one or more electronic processors 6a to 6n may be operable to execute stored instructions from their memory to assign each of the sensor-dependent, time-aligned, portioned segments of each sensor 5a to 5n and their associated first current values to one or more sensor-independent, intermediate current values that may be stored in step 205a. For ease of understanding the assigned, sensor-independent, intermediate current values may be referred to as “second current values”, or “a second set of current values”.

In embodiments, each second set of current values assigned to a portioned segment may be represented by one or more values that are less complex than the first set of current values associated with a portioned segment (i.e., the dimensions of the first set of current values representing a portioned segment are estimated and then reduced to a second set of current values). For example, a first set of current values representing each sensor-dependent portioned segment from each sensor 5a to 5n may be mapped to one or more sensor-independent, two-dimensional, intermediate current values, to name just one example of an intermediate current value during step 205. Collectively, a second set of current values (i.e., one or more sensor-independent intermediate current values) may be viewed as representing a time-aligned segment.

Accordingly, the mapping process includes functions and steps that convert values representing each sensor-dependent segment into one or more sensor-independent intermediate current values, for example.

In embodiments, because collectively all of the time-aligned portioned segments may represent a PPG waveform derived from a given sensor 5a to 5n, then, collectively, all of the so-assigned intermediate current values also represent the same PPG waveform.

It should be noted the exemplary mapping process used in step 205 receives and stores the “mapping process values” it has previously, electronically received from storage devices 4a-4n, which were generated as part of step 104 to generate the second set of current values from the first set of current values. The second set of current values may be derived from a Fourier transformation process, for example, which reduces the dimensions (and thus computational complexity) of the first set of current values.

Continuing, following the generation of the sensor-independent intermediate current values (second set of current values), in one embodiment such stored values from step 205a may then be further input and transformed into a third set of current values during step 206 (e.g., a value that represents one or more physiological states or levels stored during step 210 or physiological signals stored during step 207).

Rather than repeat the description above, suffice it to say that step 206 may generate third current values using similar processes as described with reference to steps 106a to 106n above. The processes completed during step 206 may comprise using parameters stored in 4a to 4n (using a second constrained optimization process in steps 106a to 106n) that is the reverse process of step 205 (using parameters stored in devices 4a to 4n via the first constrained optimization process in steps 105a to 105n). The second process expands the dimensions of the second set of current values such that the dimension of the third set of expanded current values now matches the dimensions of the first set of current values as those dimensions existed prior to step 205.

Some examples may benefit the reader in helping to understand the innovative real-time process or method 200 shown in FIG. 2 and primarily the details in steps 205, 205a, and 206. Suppose α and [symbol] are two time-aligned portioned segments that represent a PPG waveform from two given sensors A and B respectively. Assume one or more type of signals may be available but for the sake of explanation we assume that α from sensor A is available and we want the mapping algorithm to generate the corresponding signal [symbol] from sensor B. We can use the nomenclature m( ) to represent a mathematical function “m” and m(x) to represent values obtained from the function m( ) with inputs x; additionally, if m( ) and n( ) are two functions and x is some input, then to evaluate m(n(x)), we first use x as an input to n( ) to get values n(x) and then use values n(x) as an input to m( ) to get values m(n(x)).

Expounding on the previous discussion, recall that earlier we discussed that suppose the process of dimensionality reduction for sensor A, in step 105a, is represented by the function f( ) and the process of dimensionality expansion for sensor A, in step 106a, is represented by the function f′( ), where f′( ) is the inverse function of f( ); an inverse function is the reverse of the given function and thus f′(f(x))=x for some input x. In an embodiment, by implementing the process to complete the function f( ) the first set of values α representing a PPG waveform segment for example, from sensor A, are converted into a second values f(α) that are sensor independent. Similarly, in completing the process represented by the function f′( ) the second values f(α) may be converted into a third set of values so that f′(f(α))=α. Similarly, say that the process of dimensionality reduction for sensor B, in step 105b, is represented by the function g( ) and the process of dimensionality expansion for sensor B, in step 106b, is represented by the function g′( ), where g′( ) is the inverse function of g( ). Similarly, function g′( ) can take the second values g( [symbol] ) and convert them into set of third values so that g′(g( [symbol] ))= [symbol] . Additionally, we ensure that f(α)=g( [symbol] ) during step 104.

The intermediate values stored in storage devices 3a to 3n and the electronic processing model(s) stored in electronic storage devices 4a to 4n during step 201a may be electronically transferred to one or more electronic processors 6a to 6n.

Suppose we want to take the waveform a from sensor A and we want to transform it so that it matches [symbol] , we need to perform the operation g′(f(α)), where f(α) converts the first values α into intermediate (second values) that are sensor independent. Then we do dimensionality expand using g′( ) function by taking the second values f(α) and converting them into set of third values for sensor B. These third values are very close to if not equal to [symbol] . This process works because we ensured that f(α)=g( [symbol] ), in step 104 during the training process, that is the second values are the same for both α and [symbol] , first values which are two time-aligned portioned segments that represent a PPG waveform from two given sensors A and B at the same time. Thus, substituting f(α)=g( [symbol] ) in g′(f(α)), we get g′(f(α))=g′(g( [symbol] ))= [symbol] . The function, g′(f( )) is performed using step 205 followed by step 206 with α, the time-aligned portioned segment representing a PPG waveform from sensor A, as first values input via step 204 to obtain [symbol] as output of step 206 via the intermediate step 205a.

The mapping process comprised in steps 205, 205a and 206 discussed herein is believed to help solve the challenges involved in interpreting different data from different sensor types 5a to 5n. For example, if processes in steps 205, 205a and 206 determine that a second set of values representing a PPG waveform sensed by one of the sensors 5a to 5n (e.g., sensor 5a) closely aligns with second set of values representing a PPG waveform sensed by another one of the sensors 5a to 5n (e.g., sensor 5b), then the third set of estimated values representing the PPG waveform represented by the other sensor (sensor 5b) may be used to interpret (and reconstruct) the PPG waveforms from both sensors 5a and 5b. Said another way, the PPG waveforms from one sensor 5a may be mapped to the PPG waveforms of another sensor, such as sensor 5b. In embodiments, by a similar process, the PPG waveforms from sensor 5b may be mapped to the PPG waveforms of sensor 5a and be used to interpret (and re-construct) the PPG waveforms from sensors 5b and 5a. Still further, in embodiments, heart waveforms (PPG, ECG waveforms) from one type of sensor 5a to 5n may be mapped to the heart waveforms of another type of sensor by completing mapping processes in steps 205, 205a and 206 to aid in the re-construction and interpretation of such heart waveforms.

In an embodiment, the first, second and third sets of current values representing signal values may be stored in one or more electronic storage devices (not shown) during step 207. For a given set of sensors 5a to 5n that originally generated the different data, the stored first, second and third set of current values may comprise a set of values that may be used to correctly interpret and reconstruct physiological data (e.g., PPG, ECG waveforms) from a plurality of different sensors 5a to 5n. Further, the current values may be used to estimate and predict one or more physiological states, such as dehydration or temperature shock to name just two of the many physiological states, as explained further herein and in co-pending U.S. application No. ______ the contents of which are incorporated in full herein.

Backtracking somewhat, FIG. 2 also includes alternative steps 208, 210.

As noted above, during step 205 a first set of current values representing each sensor-dependent portioned segment from each sensor 5a to 5n may be mapped to one or more sensor-independent, two-dimensional, intermediate current values and then stored during step 205a.

The inventors discovered that the two-dimensional (and higher, i.e., “two or higher” dimensional values) although two dimensions is described here in detail) intermediate current values may be used (1) as an electronic filter, and (2) to indicate one or more physiological states (e.g., hydration, dehydration, heat stress) that can then be stored in step 210 and later used to estimate and predict one or more physiological states, such as dehydration or temperature shock to name just two of the many physiological states, as explained further herein and in co-pending U.S. application No. ______ the contents of which are incorporated in full herein without converting the values to third set of current values (i.e., without completing step 206).

In more detail, in one embodiment the one or more processors 6a to 6n may execute stored instructions retrieved from memory to generate values that may be associated with one or more estimated, physiological states or levels in step 208 based on intermediate current values, with the intermediate model having two or higher dimensions for example, without subjecting the intermediate values to additional electronic mapping processing, such as electronically expanding the dimensions of the intermediate current values for a sensor from 5a to 5n. The inventors believe that eliminating the expansion step, and performing additional processing on the expanded signal such as that in co-pending U.S. application Ser. No. ______, may significantly reduce the complexity of processing the current, different data in order to use the data to, for example, estimate current physiological states.

Referring now to FIG. 3 there is depicted one exemplary embodiment of how two or higher dimensional intermediate values can be used as an electronic filter. FIG. 3 also illustrates results from exemplary step 208.

Turning first to the filtering function/process, FIG. 3 includes two-dimensional intermediate values 300 representing PPG waveforms, for example that may be generated by the innovative mapping methods described herein. Values 300 include values 301,302 of individuals that were inferred from PPG waveforms collected from individuals while euhydrated (i.e., fully hydrated) (values 301) and while dehydrated (values 302), All of the values 300 indicate an intermediate value where each value 300 represents a PPG waveform and the intermediate two dimension values plotted on an x-y graph. In an embodiment, using the ranges of this two dimensional plot any new PPG (or ECG waveform) waveform that cannot be plotted into this input space (i.e., cannot be represented by a two-dimensional value) is considered a corrupted waveform and, therefore, can be removed (i.e., is electronically filtered).

Said another way, in an embodiment, the one or more electronic processors may be operable to electronically filter one or more of the PPG or ECG waveforms that cannot be represented by an N-dimensional value, when the generated intermediate values do not comprise N-dimensional values.

We now turn to a discussion of how two or higher dimensional intermediate values can represent one or more physiological states, again with reference to FIG. 3.

In FIG. 3 values 301 represent individuals at a euhydrated state while values 302 represent those individuals who were dehydrated. As illustrated in FIG. 3 when an individual was dehydrated there is a marked difference in the position of the intermediate values in a two-dimensional plot compared to when an individual was at rest. Thus, FIG. 3 illustrates how methods 100 and 200 may electronically determine a relative indication of one or more physiological states of an individual based on two-dimensional intermediate values, where methods 100, 200 comprise one or more of a logistic regression or classifier processing model, such as a Random Forest, K-Nearest Neighbor (KNN), Convolution Neural Network (CNN) or various machine learning or artificial intelligence processes. Though FIG. 3 is directed at hydration levels, similar two-dimensional analysis can be performed by methods 100, 200 for heat stress and blood loss as well as other physiological states.

Separate from filtering based on the use of two or higher dimensional intermediate values, the inventors further discovered that other steps or functions of the innovative mapping processes described herein also provide improved filtering or adjustment of PPG waveforms.

For example, referring to FIG. 5 there is depicted a series of waveforms 501a to 501n that illustrate how the slope of a PPG waveform may change during the time period of a single heartbeat. In an embodiment, the one or more processors 2a to 2n (and 6a to 6n) may execute stored instructions in their memories to adjust the slope of original waveform 501a from a negative slope to a positive slope (waveform 501n) in order to account for changes in the breathing pattern of an individual during steps 106a to 106n (and 206).

The waveform 501a has a negative slope whereas the wave 501n has a positive slope. Said another way, all the waves shown in FIG. 5 are embodiments of the same PPG wave (same shape) except that they include a differing amount of slope.

Suppose α and β are two “first values” representing segments of the PPG waveform shown in FIG. 5 from the same given sensor. Further assume that β=α+θ where θ indicates a slope that has been added to the α waveform. Earlier we discussed dimensionally reducing the values representing PPG segments from sensor A, in step 105a and 205 can be represented by the function f( ) and dimensionally expanding sensor A, in step 106a and 206 (for target sensor A), can be represented by the function f′( ), where f′( ) is the inverse function of f( ); an inverse function is the reverse of the given function and thus f′(f(x))=x and for some input x. f( ) converts the first set of values α and β into a second set of values f(α) and f(β). These second set of values can also be represented as sets of intermediate values f(α)=v₁, v₂, v₃, . . . , vₖ₋₁, vₖ and f(β)=u₁, u₂, u₃, . . . , uₖ₋₁, uₖ. The reader may realize that both α and β, the first set of values, represent the same waveform albeit without a slope, and with a slope, respectively, and thus ideally the second values f(α) and f(β) should have mostly the same intermediate values. In an embodiment, by realizing this relationship, the earlier constructed constrained optimization process can be completed during steps 105a to 105n to ensure f(α)=v₁, v₂, v₃, . . . , vₖ₋₁, vₖ and f(β)=u₁, u₂, u₃, . . . , uₖ₋₁, uₖ, with v₁=u₁, v₂=u₂, . . . , vₖ₋₁=uₖ₋₁, but keep the variables vₖ and uₖ, which are part of the second values, dedicated to estimating the slope of the first values α and β respectively. These variables vₖ and uₖ serve as a surrogate to the slope of the first values. Said another way, changing the slope in first values changes vₖ and uₖ. By the same analogy, when the second values f(α) and f(β) are used with dimensionality expansion function f′( ) represented in step 106a and 206 (for target sensor A), vₖ and uₖ will influence the slope in the output of 106a and 206. Of course, this is a trivial explanation as real-world PPG waveforms do not differ just on the slope but also on the actual waveform values (or shapes) and we must somehow figure out which sets of waveforms differ only by the slope and which waveforms are fundamentally different from each other (e.g.; their shapes are different). One skilled in the art and provided with enough variation of different types of waveform shapes and differing amounts of slopes can design and identify the parameters of such a constrained optimization problem that considers slope separately from the waveform values (or shape) using a database of waveforms, either automatically or manually, that differ by slope and/or shape.

An example may benefit the reader in helping to understand the innovative real-time process in step 205 that allows for automatic determination of slope and generating new waveforms with differing amount of slope. Suppose we use β=α+θ as the first current values to get second values f(β)=u₁, u₂, u₃, . . . , uₖ₋₁, uₖ which can then be used to obtain third values via the operation f′(f(β)) which are a close representation of the first values A. Previously we noted that variable uₖ which is a part of the second values is a surrogate to the slope of the first values and thus changing the slope changes uₖ. This same property can also be used to control the amount of slope in the third values f′(f(β)) by only tweaking this single variable uₖ. Example waveforms shown in FIG. 5 depicts how tweaking uₖ helps in generating waves with different amounts of positive and negative slope.

Another embodiment of the above discussed methodology is to allow for horizontal translation of the PPG waveform by adding one or more variables in the second values that control the horizontal translation of PPG waveforms and estimated by an updated constrained optimization procedure in step 105a to 105n and applied in the innovative real-time process in step 205, e.g., shifting the waveform either to the left or to the right. For the sake of brevity, we leave out the details.

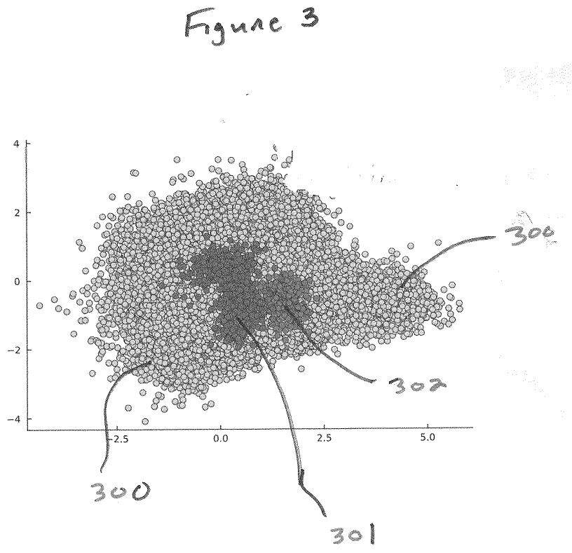

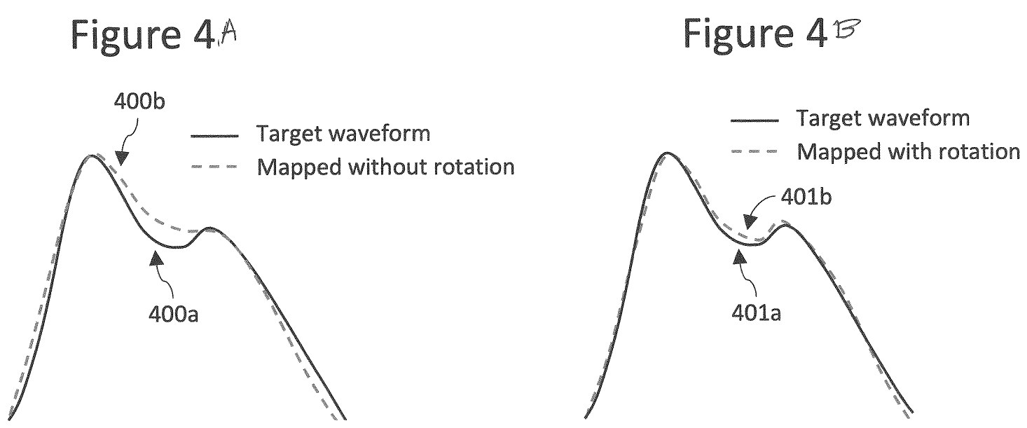

FIGS. 4A and 4B depict two sets of PPG waveforms 400a and 400b, and 401a and 401b. FIG. 4A shows an exemplary target PPG waveform, 400a, that is the waveform that ideally the mapped waveform would match is shown as a solid line, and the actual mapped waveform, 400b, is shown with a dashed line. FIG. 4A illustrates that the mapped waveform 400b somewhat resembles the target waveform 400a but does not follow the waveform too closely throughout the full length.

FIG. 4B shows the same example target PPG waveform, 401a, and the resulting mapped waveform, 401b, that includes the results of performing an electronic, rotational adjustment process. While the result of the mapped waveform in FIG. 4A is reasonable, the result of mapping with the rotational adjustment is much improved.

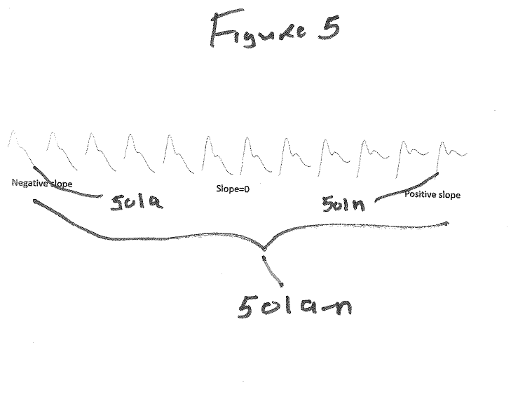

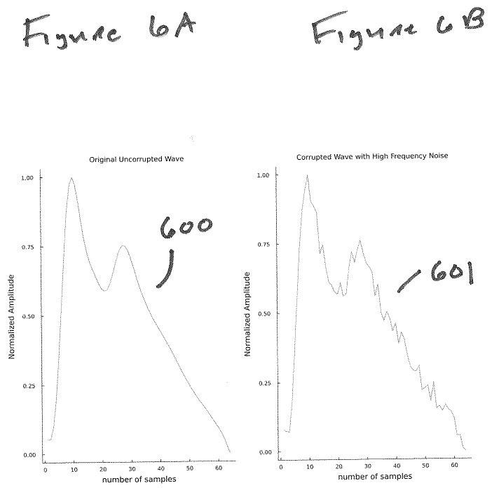

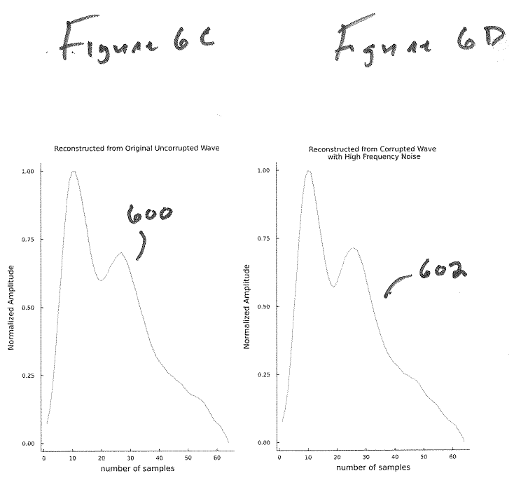

Referring now to FIGS. 6A to 6D there are depicted two exemplary PPG waveforms 600, 601 (FIGS. 6A and 6B) where waveform 600 is not corrupted and waveform 601 is corrupted, for example, by high frequency electrical noise (e.g., by motion).

In an embodiment the one or more processors 2a to 2n (and 6a to 6n) may execute stored instructions retrieved from memory to receive corrupted waveform 601 and then complete the mapping process of methods 100 (and 200).

When such a corrupted PPG waveform is input into the innovative mapping processes (with, or without any pre-filtering by steps 102, 202) the mapping processes functions as an electronic filter to remove corrupted data (values).

For example, FIG. 6D depicts waveform 602 that was generated by inputting corrupted waveform 601 into the innovative mapping processes described herein.

As depicted, the shape of the reconstructed waveform 602 closely resembles the original uncorrupted signal 600 (in FIG. 6A or 6C). The intermediate values (in a 2-dimensional space, not shown) were very close to each other for both waveforms 600, 602 (e.g., −1.89 vs −1.96 for x-axis, −1.35 vs −1.53 for y-axis.

The mapping of the original noisy waveform to a reduced dimension intermediate domain model accommodates variation in the signal values such that when the intermediate domain waveform is expanded it will be mapped to a waveform that does not have the noise. This filtering is applicable when mapping from a sensor to itself (i.e., Sensor A to Sensor A) or when mapping from a Sensor to another sensor (i.e., Sensor A to Sensor B). This filtering is also applicable when mapping from a sensor to the intermediate domain, such as for determining physiological values (i.e., Sensor A to intermediate domain).

### Experiments

Experiments implementing exemplary method 100 were completed. Data was collected using two different scenarios: (1) individuals undergoing simulated hemorrhaging, and (2) individuals exercising under various heat and dehydration conditions.

Physiological data was collected from 62 individuals using two finger sensors. The first sensor was made by one manufacturer, while the second sensor was made by different manufacturer.

Measurements were taken prior, during, and immediately following periods of Lower Body Negative Pressure (LBNP). Individuals were subjected to increasing levels of LBNP, until the onset of hemodynamic decompensation.

For example, an individual's lower body was placed in a sealed, LBNP chamber to isolate the lower body from the surrounding environment. Thereafter, air was progressively removed from the chamber via a vacuum pump which lowers the air pressure in the chamber thus drawing blood away from the upper part of the body. This simulates a loss of blood volume and was used to simulate hemorrhaging.

During the lowering of the pressure, continuous PPG signals were recorded by both finger sensors of an individual at the same instant of time. Data representing the PPG signals was separated into individual heartbeat waveforms and electronically stored in a database (such as storage devices 1a to 1n discussed above). Signals representing over 60,000 PPG waveforms were measured at the same time using both sensors.

PPG data from the waveforms was electronically adjusted to remove corrupted data as in step 102. The PPG data were then electronically mapped to one or more intermediate values as in steps 103 and the mapping process 104.

It was observed that the intermediate values based on the PPG data measured by each sensor at the same time instant were similar, if not the same value.

It was additionally observed that a PPG waveform that was sensed using the first sensor was correctly, electronically reconstructed using intermediate values that were associated with the PPG waveform sensed by the second sensor and vice-versa.

To further evaluate the mapping process, a held out cross-validation approach was implemented. In more detail, a random sample of 10% of the signals associated with sensed PPG waveforms were stored for validation purposes.

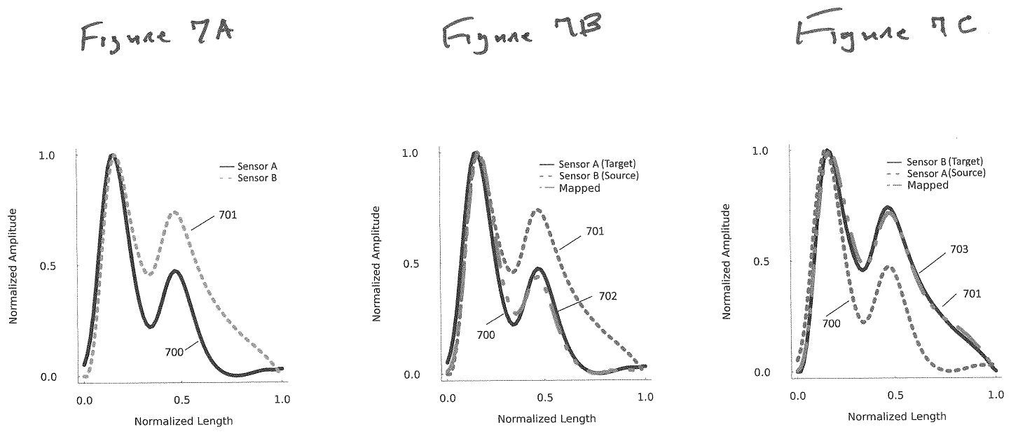

Referring to FIGS. 7A to 7C there is depicted randomly selected validation PPG waveforms 700, 701, where one PPG waveform 700 is derived from the first sensor and the second waveform is derived from the second sensor 701 at the same instant of timer. As shown, the waveforms have distinctive and differing shapes.

FIG. 7B depicts the same two waveforms 700, 701 and in addition a third waveform 702. The third waveform 702 was output from an innovative mapping processes described herein, where waveform 702 represents a reconstruction (or mapping) of waveform 701 to waveform 700. As shown, waveform 702 is very similar to waveform 700.

FIG. 7C depicts the same two waveforms 700, 701 and in addition a third waveform 703. The third waveform 703 was output from the innovative mapping processes described herein, where waveform 703 represents a reconstruction (or mapping) of waveform 700 to waveform 701. As shown, waveform 703 is very similar to waveform 701.

We claim:

1. A method for mapping physiological data comprising;
electronically receiving different physiological data, the data representing one or more of sensed photoplethysmography (PPG) or electrocardiogram (ECG) waveforms;
electronically portioning the different physiological data into segments;
electronically generating one or more sensor-dependent values representing each segment;
electronically assigning each of the sensor-dependent values to an intermediate value;
further electronically assigning each of the intermediate values to a value that represents one or more physiological states or levels or physiological signals.

2. The method of claim 1, wherein the different physiological data comprises physiological signal data sensed by sensors that are classified differently.

3. The method of claim 1, wherein the different physiological data comprises physiological signal data sensed by sensors positioned at different specific locations on an individual's body.

4. The method of claim 1, wherein the different physiological data comprises physiological signal data sensed by sensors that use different specific, operating characteristics.

5. The method of claim 1, wherein the different physiological data comprises physiological signal data sensed by sensors that output data using different, specific formats or protocols.

6. The method of claim 1, wherein electronically portioning the different physiological data into segments further comprises electronically portioning the different physiological data into segments of equal length.

7. The method of claim 1, wherein electronically portioning the different physiological data into segments further comprises electronically portioning one or more of the portioned segments into a physiological signal segment having a different length than another portioned segment.

8. The method as in claim 1 wherein the intermediate values comprise a two or higher dimensional value, and the method further comprises electronically filtering one or more of the PPG or ECG waveforms that cannot be represented by the two or higher dimensional value.

9. The method as in claim 1 wherein the intermediate values comprise a two or higher dimensional value, and the method further comprises electronically determining a relative indication of one or more physiological states of an individual based on the two or higher dimensional value.

10. The method as in claim 1 further comprising electronically applying a rotational or slope adjustment to the PPG or ECG waveforms to account for physiological changes.

11. The method as in claim 1 further comprising electronically filtering one or more of the PPG or ECG waveforms that cannot be represented by an N-dimensional value when the generated intermediate values do not comprise N-dimensional values.

12. An electronic device for mapping physiological data comprising;
one or more electronic processors operable to execute instructions stored in one or more electronic memories to:
electronically receive different physiological data, the data representing one or more of sensed photoplethysmography (PPG) or electrocardiogram (ECG) waveforms;
electronically portion the different physiological data into segments;
electronically generate one or more sensor-dependent values representing each segment;
electronically assign each of the sensor-dependent values to an intermediate value;
further electronically assign each of the intermediate values to a value that represents one or more physiological states or levels or physiological signals.

13. The electronic device of claim 12, wherein the different physiological data comprises physiological signal data sensed by sensors that are classified differently.

14. The electronic device of claim 12, wherein the different physiological data comprises physiological signal data sensed by sensors positioned at different specific locations on an individual's body.

15. The electronic device of claim 12, wherein the different physiological data comprises physiological signal data sensed by sensors that use different specific, operating characteristics.

16. The electronic device of claim 12, wherein the different physiological data comprises physiological signal data sensed by sensors that output data using different, specific formats or protocols.

17. The electronic device of claim 12, wherein the one or more electronic processors are further operable to execute instructions stored in one or more electronic memories to electronically portion the different physiological data into segments of equal length.

18. The electronic device of claim 12, wherein the one or more electronic processors are further operable to execute instructions stored in one or more electronic memories to electronically portion the different physiological data into one or more physiological signal segments having a different length than another portioned segment.

19. The electronic device of claim 12, wherein the intermediate values comprise a two or higher dimensional value, and the one or more electronic processors are further operable to execute instructions stored in one or more electronic memories to electronically filter one or more of the PPG or ECG waveforms that cannot be represented by the two or higher dimensional value.

20. The electronic device of claim 12, wherein the intermediate values comprise a two or higher dimensional value, and the one or more electronic processors are further operable to execute instructions stored in one or more electronic memories to electronically determine a relative indication of one or more physiological states of an individual based on the two or higher dimensional value.

21. The electronic device of claim 12, wherein the one or more electronic processors are further operable to execute instructions stored in one or more electronic memories to further electronically apply a rotational or slope adjustment to one or more of the PPG or ECG waveforms to account for physiological changes.

22. The electronic device of claim 12 wherein the one or more electronic processors are further operable to execute instructions stored in one or more electronic memories to further electronically filter one or more of the PPG or ECG waveforms that cannot be represented by an N-dimensional value when the generated intermediate values do not comprise N-dimensional values.
